(19) United States
(12) Patent Application Publication
(10) Pub. No.: US 2005/0195366 A1
(43) Pub. Date: Sep. 8, 2005

(54) Fragrance delivery for multimedia systems

(76) Inventors: Raymond K. Selander, Hopewell Junction, NY (US); Clint Dee Winton Brooks, Sea Bright, NJ (US); Lewis Michael Popplewell, Morganville, NJ (US)

Correspondence Address: INTERNATIONAL FLAVORS & FRAGRANCES INC., 521 WEST 57TH ST, NEW YORK, NY, 10019, US

(21) Appl. No.: 10/791,923

(22) Filed: Mar. 3, 2004

(51) Int. Cl. A61L 9/04

(52) U.S. Cl. 352/85

(57) ABSTRACT

Apparatus and methods for fragrance sensory stimulation connected to a multimedia source that has at least one audiovisual signal connected to an audiovisual display, and also has fragrance information synchronized with the audiovisual signal is disclosed. In preferred embodiments, a fragrance generator processes the fragrance information into a fragrance signal and at least one fragrance control system accepts a fragrance signal as an input, and generates a control signal and a burst of compressed gas. In turn, a fragrance delivery system preferably comprising a plurality of fragrance chemicals in communication with the burst of compressed gas selectively volatilizes the fragrance chemicals so that a short burst of fragrance is sensed by the subject. Preferably, this short burst is followed by a purging burst that clears the fragrance so that another subsequent fragrance is easier to sense. The compressed gas may be generated by a source of compressed gas or a compressor. In certain embodiments, the fragrance delivery system comprises an indexing turret containing a plurality of ports, each port containing one of said plurality of fragrance chemicals. In other preferred embodiment, a multiport micro-valve or array of such valves contains an absorbent material impregnated with a fragrance in each port.

The present invention relates to fragrance delivery systems, and more particularly to fragrance delivery systems that are synchronized with audio and/or visual presentations to add fragrance to a display of sights and/or sounds.

### BACKGROUND OF THE INVENTION

The “experience” of audiovisual entertainment has progressed from silent films and monaural recordings to today's visually stunning digital images and advances in sound reproduction such as the “surround sound” found in both commercial theatres and homes. Moreover, current computer-based games and games played on gaming platforms employ the same advanced graphics and sound qualities found in film or video. These technologies create an audiovisual “experience” that immerses the user in a film or game like never before, stimulating sight, sound and even tactile sensations through deep bass vibrations provided by subwoofers and tactile feedback provided by some game controllers. There remains, however, one form of sensory perception that is not stimulated—the sense of smell.

Numerous prior art systems have attempted to provide a scent to the environment that compliments or correlates to an audiovisual stimulus. Examples would be the smell of burning rubber coordinating with the screech of tires, or the scent of flowers when a corresponding image appears. Prior art systems have primarily been directed to introducing fragrances to large environments, such as an entire theater. These systems have by and large suffered from the problem that a fragrance will linger long after the coordinating audiovisual input has changed, and may in fact be difficult to replace with another scent as the scenes change, the typical result being a mixture of several fragrances that become an indistinct muddle.

None of these prior art devices, however, provides a useful and commercially viable system for fragrance delivery to enhance an audiovisual presentation. Therefore, there remains a long-felt yet unmet need for providing it and would therefore be desirable to provide. It would further be desirable to provide such improvements in a manner that permitted their application across a variety of situations and that permitted their implementation in a cost-effective manner.

### SUMMARY OF THE INVENTION

Accordingly, it has now been found that these and other shortcomings of the prior art can be overcome by providing an apparatus for fragrance sensory stimulation that is connected to a multimedia source that has at least one audiovisual signal connected to an audiovisual display, and also has fragrance information synchronized with the audiovisual signal. In preferred embodiments of the present invention, a fragrance generator processes the fragrance information into a fragrance signal and at least one fragrance control system accepts a fragrance signal as an input, and generates a control signal and a burst of compressed gas. In turn, a fragrance delivery system preferably comprising a plurality of fragrance chemicals in communication with the burst of compressed gas, selectively volatilizes the fragrance chemicals so that a short burst of fragrance is sensed by the subject. In certain embodiments, the fragrance delivery system comprises an indexing turret containing a plurality of ports, each port containing one of said plurality of fragrance chemicals. In other preferred embodiment, a multiport micro-valve or array of such valves contains an absorbent material impregnated with a fragrance in each port.

In certain embodiments, the system is constructed as two subassemblies, in which a transmitter is connected to the fragrance generator and a receiver is connected to the fragrance creation system, and the transmitter sends the fragrance signals to the receiver to control the creation of synchronized bursts of fragrance, most preferably provided by a compressor or source of compressed gas regulated by a microprocessor and including a high-pressure cutoff sensor so that a short burst of pressurized air having a duration of less than five seconds is created. In certain preferred embodiments of the apparatus of the present invention, the fragrance control system is mounted on a user's body, while in other preferred embodiments, the fragrance control system is mounted on a chair. Similarly, in certain preferred embodiments of the apparatus of the present invention, the fragrance delivery system is mounted on a user's body, while in other preferred embodiments, the fragrance control system is mounted on a chair.

The present invention thus provides a fragrance producing system synchronized to an audiovisual medium that preferably includes a fragrance delivery system for providing a short burst of air containing one of a plurality of fragrance chemicals to an individual user coordinated by receiving and processing a signal contained within the audiovisual medium that has been transmitted to a fragrance creation system in which the signal activates a compressor or releases a source of compressed gas that selectively volatilizes the fragrance chemical, wherein the plurality of fragrance chemicals are disposed within an apparatus that selectively mixes one of the plurality with a burst of pressurized air or compressed gas. Most preferably, the signal contained within the audiovisual medium is transmitted via and RF transmitter to an RF receiver connected to the fragrance creation system, and the apparatus that selectively mixes one of the plurality with a burst of pressurized air generated by the compressor or burst of a compressed gas comprises a turret with a plurality of ports, and each of the ports includes an absorbent material and a fragrance chemical. Alternatively, the apparatus that selectively mixes one of the plurality with a burst of pressurized air or compressed gas comprises an array of micro-valves, and each of the micro-valves includes an absorbent material and a fragrance chemical.

Finally, the present invention also discloses methods of synchronizing a fragrance stimulus to a user with one or more audio and visual stimuli by providing a fragrance track to an audiovisual medium to provide a coded signal that correlates to a type of fragrance desired to be released at a pre-determined time, and then determining the divisions within the medium in which fragrance is to be delivered and creating a sequence of fragrances. A device having the requisite number of ports containing a sufficient variety and quantity of fragrance to correspond to the sequence of fragrances is provided and loaded with the fragrance. Next, a signal is processed to activate a compressor or release a burst of compressed gas and connect the device with a burst of compressed gas so that the correct fragrance chemical is volatilized when the burst reaches the fragrance chemical. In certain embodiments the step of providing a fragrance track comprises programmatic methods direct the system to generate a scent for a pre-determined brief period of time.

### BRIEF DESCRIPTION OF THE DRAWINGS

FIG. 1 is a block diagram illustrating a preferred embodiment of the present invention.

FIG. 2 is a partially schematic, partially perspective view of a preferred embodiment of a fragrance creation system used in the system shown in FIG. 1.

FIG. 3 is a partially schematic, partially perspective view of a preferred embodiment of the delivery creation system used in the system shown in FIG. 1.

### DETAILED DESCRIPTION OF THE PREFERRED EMBODIMENTS

The implementation of the present invention is in several preferred embodiments, discussed below, along with several illustrative examples. The embodiments of the invention described below are provided for the purpose of understanding the invention and are not meant to be limiting.

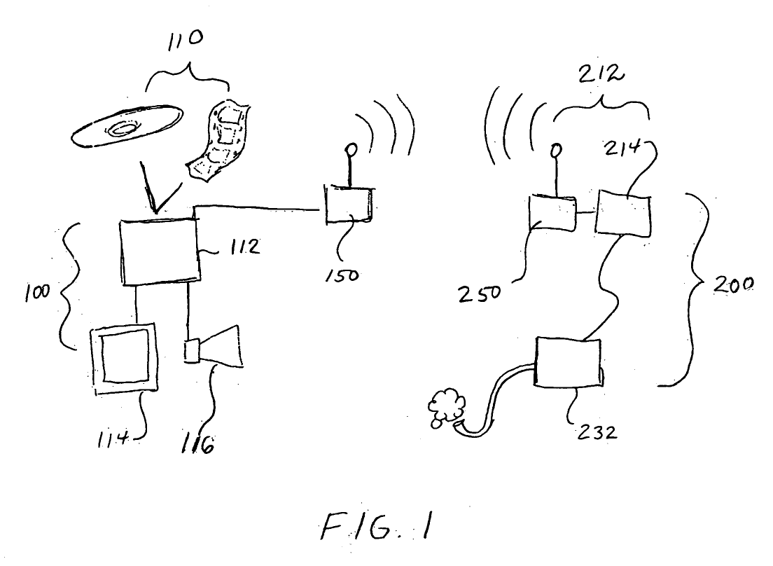

Referring now to FIG. 1, a first embodiment of a system employing the present invention is illustrated. There are two main sub-systems that make up this embodiment, namely, the fragrance generator 100 and the fragrance delivery system 200, each of which is discussed in further detail below. In the preferred embodiment illustrated in FIG. 1, the fragrance generator 100 and the delivery system 200 are preferably connected by a wireless system comprising a transmitter 150 and a receiver 250 which are most preferably RF devices, while in other embodiments, such devices and subassemblies can be connected by Infrared (IR) transmission or wires, or integrated into a single unit. In preferred embodiments of the present invention, a converter allows a PC gaming program to transmit a synchronous fragrance selection from a PC's COMM Port. Typically, although not necessarily, this converter accepts a special COMM command, representing the “fragrance track” described below and converts it to an RF Transmitting module. The RF Transmitting module operates at 433 MHz and transmits to distances of 150 feet. Alternatively, a converter can be used to allow a PC gaming program to transmit a synchronous fragrance selection from a PC's COMM Port. In this embodiment, the converter accepts a special COMM command, representing the fragrance track and converts it to a newly programmed microprocessor which transmits the IR signal through a photodiode. The IR converter has a shorter range than the RF converter, with a maximum distance of 65 feet.

The fragrance generator 100 is comprised of a medium 110 and a media player 112. As known in the art, the medium 110 and media player 112 can be any one of a number of systems, either digital, analog or some combination of such systems, that contains information and converts the information into a signal that can be used for display. Thus; the medium 110 will comprise one or more “tracks” such as a video track, a sound track and, in accordance with the present invention, a fragrance track. The media player 112 will read or process the medium 100 and create signals that can be displayed by devices such as a visual display 114 and an audio display 116. However, in accordance with the present invention, the scent track described above is transmitted to and processed by a fragrance control system 212 that may or may not be integral with the media player 112. As discussed in further detail below and well known in the art, there are a number of systems that contain a scent “medium” (not illustrated in FIG. 1) that can be “played” by the fragrance control system 212 to control the selective release of one or more scents in coordination with the information being provided to the visual display 114 and audio display 116. The coordination of emitting various scents during playback of a video, or during the playing of a video game is disclosed in U.S. Pat. No. 6,654,664—Manne and in U.S. patent application Publications 2001/0008611 and 2002/0036358, none of which are admitted to be prior art to the present invention.

As illustrated in FIG. 1, in certain embodiments of the present invention, the fragrance control system 212 is divided into two sections, although they may be physically integrated if desired. In accordance with one aspect of the present invention, the fragrance creation system 212 provides short bursts of synchronized fragrances coordinated with audio or visual information on a real-time basis. A first section of the fragrance control system 212 is the fragrance creation system 214.

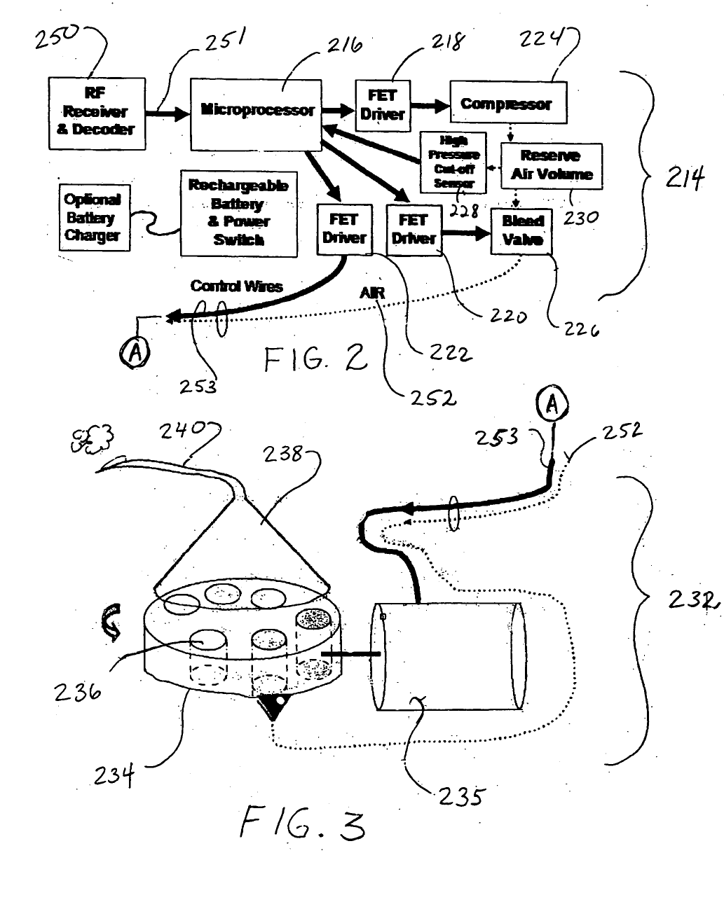

Referring now to FIG. 2, a block diagram of one preferred embodiment of a fragrance creation system 214 is illustrated. The fragrance creation system 214 can be either connected directly to the fragrance generator 100, or as described above, may be connected via an RF transmitter 150 and receiver 250 as described above with reference to FIG. 1, or connected via an IR transmitter 150 and receiver 250 as described above with reference to FIG. 1. In such an embodiment illustrated in FIG. 2, the RF receiver 250 will include a decoder for RF signal drivers. In any event, a fragrance signal 251 is provided to a microprocessor controller 216 which in turn controls a set of FET drivers 218, 220, 222 that control, respectively, a compressor 224, a bleed valve 226 and other portions of the device. In certain preferred embodiments, the compressor 224 is replaced with a source of compressed gas such as carbon dioxide, nitrogen, oxygen, air or other gases that are safe for inhalation. Using a source of compressed gas would be particularly desirable in situations where a compressor is unduly expensive, noisy, generates too much heat or has excessive power requirements. An additional high-pressure cutoff sensor 228 monitors a reserve air volume 230 and the bleed valve 226. In operation, the microprocessor controller 216 activates the compressor 224, or admits a short burst from a compressed gas source 224 as described above. When the upper limit of pressure is achieved, the high pressure cutoff sensor 228 signals the microprocessor to deactivate the compressor or cease the supply of compressed gas, thus creating and maintaining a pressurized system so that when appropriate a short burst of pressurized air is available to be conducted elsewhere in the system. This system can either be battery powered or wired to a current source. In operation, the microprocessor converts the fragrance signal 251 into a signal that controls a regulated flow of air 252 created by the compressor or source of compressed gas 224 and a control signal 253, both of which are carried to a fragrance delivery system 232, discussed in detail below with relation to FIG. 3.

As will be appreciated by those of skill in the art, the system described with relation to FIG. 2 is relatively easy to construct and will be a relatively small-scale unit, thereby permitting devices made in accordance with this aspect of the present invention to be integrated into a compact unit at a relatively low cost to enable economic mass production and widespread implementation. Moreover, such a construction will permit the fragrance creation system 214 to be constructed in embodiments that can be body-worn, e.g., on a waist belt, or conveniently and unobtrusively mounted to a theatre chair, airline or vehicle passenger seat, or a seat in the home.

Referring now to FIG. 3, a partially schematic, partially perspective view of a preferred embodiment of a fragrance delivery system 232 is illustrated. As discussed above with reference to FIG. 2, a burst of compressed gas 252 and an electrical control signal 253 are carried from the fragrance creation system 214. The burst of compressed gas 252 is connected via a conduit such that it flows into and through a turret 234 that houses a plurality of fragrance chemicals 236 that are volatilized when the burst of compressed gas 252 enters the turret 234. In the preferred embodiment illustrated, the turret 234 includes a plurality of ports or wells 236 that contain an absorbent material that is impregnated or “charged” with a particular scent. The electrical control signal 253 controls a solenoid 235 that indexes the turret to a position that corresponds to a particular port 236. Depending upon the use, e.g., commercial or home use, the ports 236 may be accessible so they can be recharged or reloaded with fragrance chemicals, or the entire turret may be a disposable structure that is replaced each time the system is used, or replaced after a specified number of viewings of a film or sessions of game play. In certain other preferred embodiments, the turret 236 can be replaced by an array of micro-valves, each disposed over a port 236 containing fragrance chemical, and the fragrance is volatilized upon opening of the valve by the signal 235 and the passage of the burst of compressed gas 252 through the open orifice of the valve.

Still referring to FIG. 3, as the burst of compressed gas passes through the turret 236, it is collected by a manifold hood 238 which in turn conducts the airflow, which now carries the selected scent, to a nosepiece 240 or similar structure that directs the fragrance to the nostrils of a single user. The use of a nosepiece and related apparatus to conduct a flow of scented air to a wearer is known in the art, for example, U.S. patent application Publication 2004/0003812, which is not admitted to be prior art to the present invention, discloses a portable fragrance control device that releases scent directly into a user's nose via diffusion. The device disclosed is structurally similar to the headsets commonly used for listening and/or speaking. The fragrance delivery system 232 is thus controlled by the signal provided by the fragrance creation system 214 and “powered” by the compressor or supply of compressed gas 224 that forms part of that same subsystem. Preferably, the fragrance delivery system 232 is-constructed to be lightweight and can be either worn on the body or integrated into the headrest of a chair, but in either case it is preferred that the nosepiece 240 be disposed proximate the wearer's or user's nose so that the fragrance is conducted directly to the individual and does not permeate the environment generally. Moreover, as mentioned above, it is further desirable that the fragrance delivery system 232 emits short bursts of fragrance at the appropriate synchronized time to enhance the experience of a game, movie or similar audiovisual presentation.

In certain preferred embodiments, the present invention will also selectively provide a purging burst to provide complete separation of scents. Thus, after a delivery of any fragrance, a fragrance signal 251 to the microprocessor controller 216 will again send a flow of air through the system, however in “purge mode” no fragrance will be volatilized, and a clean burst of air clears the airflow supplied to the user.

Thus, in operation, those of skill in the art will appreciate that a fundamental requirement of the present invention is that the medium contain an additional “track” that is designed to provide a coded signal that correlates to the type of scent desired to be released at the particular temporal point in the audio or visual presentation. Alternatively, programmatic methods could be employed, e.g., a short algorithm that directs the system to generate a scent upon intervals, e.g., generating short bursts (for example, less than three seconds) of pine scent every thirty seconds during prolonged scenes in an evergreen forest. Such signals, whether part of a “track” or programmed are easily integrated into various forms of media, such as digital discs (CDs, DVDs, etc.), game cartridges, the magnetic tracks found on commercial theatre film, videotape and audiotape, and various other forms of magnetic media. Typically, the numbers of scenes or similar divisions within the medium in which fragrance will make an impact or otherwise be appropriate are identified and a sequence of fragrances is created. A turret or array of valves having the requisite number of ports containing a sufficient variety and quantity of fragrance to correspond to the sequence of fragrances is designed and provided. In operation, the system is loaded with the correct turret or valve array and the user is positioned so the nosepiece described above will transmit fragrance to the wearer in a synchronized fashion. The “fragrance track” provides a signal that is processed to cause a burst of gas to flow and indexes the turret so that the correct fragrance chemical is volatilized when the burst of air reaches the fragrance chemical.

Upon review of the foregoing, numerous adaptations, modifications, and alterations will occur to the reviewer. These will all be, however, within the spirit of the present invention. Accordingly, reference should be made to the appended claims in order to ascertain the true scope of the present invention.

1. An apparatus for fragrance sensory stimulation comprising:
a multimedia source comprising at least one audiovisual signal connected to an audiovisual display, and fragrance information synchronized with the audiovisual signal;
a fragrance generator for processing said fragrance information into a fragrance signal;
at least one fragrance control system that accepts a fragrance signal as an input, and generates a control signal and a burst of compressed gas; and
a fragrance delivery system comprising a plurality of fragrance chemicals in communication with the compressed gas, whereby at least one of said fragrance chemicals is volatilized and the volatilized fragrance is collected and sensed by a subject.

2. The apparatus for fragrance sensory stimulation according to claim 1, wherein the fragrance delivery system comprises an indexing turret containing a plurality of ports, each port containing one of said plurality of fragrance chemicals.

3. The apparatus for fragrance sensory stimulation according to claim 2, wherein the multiport valve contains an absorbent material impregnated with a fragrance in each port.

4. The apparatus for fragrance sensory stimulation according to claim 1, wherein the delivery system comprises a miniature multiport valve.

5. The apparatus for fragrance sensory stimulation according to claim 1, further comprising a transmitter connected to the fragrance generator and a receiver connected to the fragrance creation system, whereby the transmitter sends the fragrance signals to the receiver.

6. The apparatus for fragrance sensory stimulation according to claim 1, wherein the fragrance control system comprises a source of compressed gas regulated by a microprocessor.

7. The apparatus for fragrance sensory stimulation according to claim 6, wherein the source of compressed gas is a compressor.

8. The apparatus for fragrance sensory stimulation according to claim 6, wherein the source of compressed gas is a vessel.

9. The apparatus for fragrance sensory stimulation according to claim 6, wherein the source of compressed gas contains a gas selected from the group comprising: carbon dioxide, air, nitrogen, and oxygen.

10. The apparatus for fragrance sensory stimulation according to claim 6, further comprising a high-pressure cutoff sensor.

11. The apparatus for fragrance sensory stimulation according to claim 1, wherein the short burst of pressurized gas is a burst of less than five seconds duration.

12. The apparatus for fragrance sensory stimulation according to claim 1; wherein the burst of compressed gas is followed by a purging burst of compressed gas that clears fragrance chemicals from the apparatus.

13. The apparatus for fragrance sensory stimulation according to claim 1, wherein the fragrance control system is mounted on a user's body.

14. The apparatus for fragrance sensory stimulation according to claim 1, wherein the fragrance control system is mounted on a chair.

15. The apparatus for fragrance sensory stimulation according to claim 1, wherein the fragrance delivery system is mounted on a user's body.

16. The apparatus for fragrance sensory stimulation according to claim 1, wherein the fragrance control system is mounted on a chair.

17. A fragrance producing system synchronized to an audiovisual medium comprising: a fragrance delivery system for providing a short burst of compressed gas containing one of a plurality of fragrance chemicals to an individual user coordinated by receiving and processing a signal contained within the audiovisual medium that has been transmitted to a fragrance creation system in which the signal releases a burst of compressed gas that selectively volatilizes the fragrance chemical, wherein the plurality of fragrance chemicals are disposed within an apparatus that selectively mixes one of said plurality with a burst of compressed gas.

18. The fragrance producing system according to claim 17, wherein the signal contained within the audiovisual medium is transmitted via and RF transmitter to a RF receiver connected to the fragrance creation system.

19. The fragrance producing system according to claim 17, wherein the apparatus that selectively mixes one of said plurality with a burst of compressed gas comprises a turret with a plurality of ports, and each of said ports includes an absorbent material and a fragrance chemical.

20. The fragrance producing system according to claim 17, wherein the apparatus that selectively mixes one of said plurality with a burst of pressurized gas comprises an array of micro-valves, and each of said micro-valves includes an absorbent material and a fragrance chemical.

21. The fragrance producing system of claim 17, further wherein-said compressed gas selectively creates a purging airflow, whereby said purging burst of compressed gas clears fragrance chemicals from the apparatus.

22. The fragrance producing system according to claim 17, wherein the signal contained with the audiovisual medium is transmitted via an infrared transmitter to an infrared receiver connected to the fragrance creation system.

23. A method of synchronizing a fragrance stimulus to a user with one or more audio and visual stimuli, comprising the steps-of:
providing a fragrance track to an audiovisual medium to provide a coded signal that correlates to a type of fragrance desired to be released at a pre-determined time;
determining the divisions within the medium in which fragrance is to be delivered and creating a sequence of fragrances;
providing a device having the requisite number of ports containing a sufficient variety and quantity of fragrance to correspond to the sequence of fragrances;
loading the device with the fragrance; and
providing a signal that is processed to activate a source of compressed gas and connect the device with a burst of compressed gas that the correct fragrance chemical is volatilized when a burst of air reaches the fragrance chemical.

24. The method according to claim 23, wherein the step of providing a fragrance track comprises programmatic methods directing the system to generate a scent for a pre-determined brief period of time.

25. The method according to claim 23, wherein the step of connecting the device with a burst of compressed gas comprises indexing a turret.

26. The method according to claim 23, wherein the step of connecting the device with a burst of compressed gas comprises actuating one of an array of valves.

27. The method according to claim 23, further comprising the step of providing a signal to activate the source of compressed gas to purge any fragrance chemical within the device.
